(19) United States
(12) Plant Patent Application Publication
(10) Pub. No.: US 2003/0115650 P1
(43) Pub. Date: Jun. 19, 2003

(54) Clematis plant named ' Vanso'

(76) Inventor: Frans C.M. van Haastest, Holland (NL)

Correspondence Address: CHRISTIE, PARKER & HALE, LLP, 350 WEST COLORADO BOULEVARD, SUITE 500, PASADENA, CA, 91105, US

(21) Appl. No.: 10/029,663

(22) Filed: Dec. 18, 2001

(51) Int. Cl. A01H 5/00

(52) U.S. Cl. PLT/228

(57) ABSTRACT

Described as a new variety of Clematis which produces fully doubled flowers of attractive coloration.

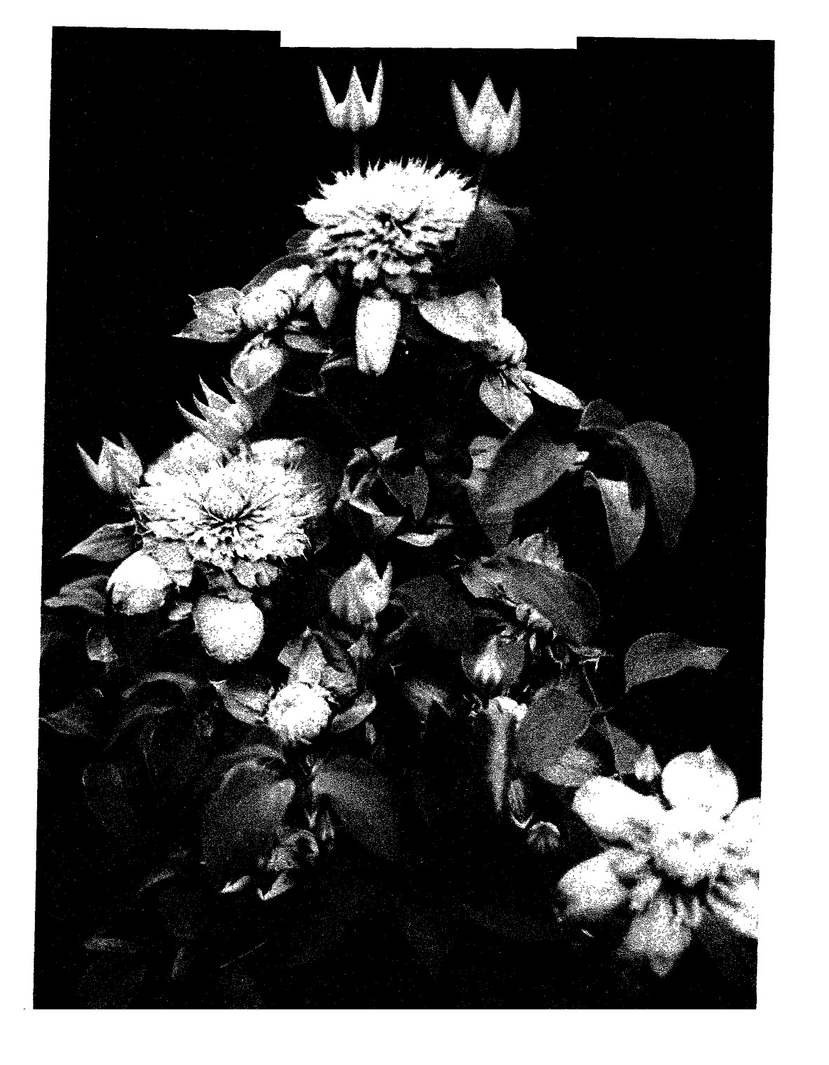

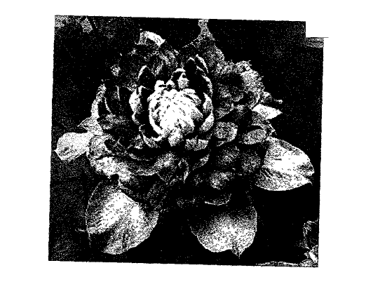

### BACKGROUND OF THE INVENTION

The present invention relates to a new Clematis plant having the varietal denomination ‘VANSO’. ‘VANSO’ was discovered as a natural branch mutation of the variety ‘MRS. CHALMONDELEY’, not known to be patented.

### SUMMARY OF THE INVENTION

The new variety is a Clematis plant with a climbing habit distinguished by production of fully double flowers. Asexual reproduction of the new variety as performed by softwood cuttings in Boskoop, the Netherlands has confirmed that the distinguishing characteristics of the new variety are transmitted through succeeding generations.

### COMPARISON WITH PARENT

The new variety differs from its parent, ‘MRS. CHALMONDELEY’, in producing flowers which are fully double; whereas flowers of the parent variety are single.

### BRIEF DESCRIPTION OF ILLUSTRATIONS

The accompanying illustrations depict specimen flowers of the new variety in color as true as possible in color photographic illustrations of this nature.

One figure shows the plant of the new variety with a cluster of flowers; and

The other figure shows a close up of the flower of the new variety.

### DETAILED DESCRIPTION OF THE NEW VARIETY

The following is a description of the new variety taken from plants grown out-of-doors in Boskoop, the Netherlands during the month of June. Color designations used herein are from the ROYAL HORTICULTURAL SOCIETY COLOR CHART (“RHSC”).

Plant: The plant grows in a climbing habit and attains a height of slightly more than 2 M. with vigorous growth.

Foliage:

Size.—About 5 cm. to 8 cm. long and up to about 4.8 cm. wide.

Quantity.—About 6 leaves (3 pairs of opposite leaves) per about 15 cm. stem.

Color.—Immature foliage Upper side: Between 141A and 143A Underside: Between 146A and 146B Mature foliage Upper side: Near 137A Under side: Between 137B and 137C.

Shape.—Trifoliolate — leaflets are ovate to narrow ovate.

Texture.—Upper side: Smooth Under side: Smooth.

Veination.—Anastomosing.

Edge.—Smooth with no serration observed.

Petiole color.—Top surface: Near 166A Under side: Between 143A and 141B.

Stipules.—No stipples observed.

Auricles.—No auricles observed.

Rachis.—Smooth.

Wood:

Color.—Near 143A.

Bark.—Bark has short pale greyish hairs.

Flower: Not for use as a cut flower

Color.—Clear blue double flowers.

Winter Hardiness: Winter hardy in USDA Zone 5

Resistance to Disease: Good, no more susceptible to disease than its parent variety or other similar varieties.

Breeding: Asexual reproduction was started from softwood cuttings during the summer months in tunnels. Natural mutation was observed when one branch of the parent variety flowered with double flowers. This mutation branch was then propagated by softwood cuttings.

Stamens, Filaments & Anthers:

Arrangement.—Flowers are completely double, so no stamens, styles, etc. are visibly present. No seed formation observed.

I claim:

1. A new Clematis plant of the variety substantially as shown and described.
